(19) United States
(12) Patent Application Publication
(10) Pub. No.: US 2006/0263346 A1
(43) Pub. Date: Nov. 23, 2006

(54) Combined pharmaceutical preparations for the treatment of cancer, containing glutaminase and antineoplastic anthracyclines or platinum compounds

(75) Inventors: Frank Leenders, Berlin (DE); Seifert Wenke, Berlin (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Medical Enzymes AG, Berlin (DE)

(21) Appl. No.: 10/560,317

(22) PCT Filed: Jun. 11, 2004
(86) PCT No.: PCT/EP04/06320

§ 371 Date: Jul. 13, 2006

(30) Foreign Application Priority Data
Jun. 11, 2003 (DE) 103 26 821.9

(51) Int. Cl. A61K 38/48; A61K 31/282; A61K 31/704; A61K 33/24

(52) U.S. Cl. 424/94.63; 424/649; 514/34; 514/492

(57) ABSTRACT

The invention concerns combined pharmaceutical preparations which inhibit abnormal growth of tumor cells. The combined preparations comprise as active substances compounds having glutaminase activity in combination with certain antineoplastic agents. The invention concerns in particular combined preparations of compounds having glutaminase activity and cytostatic compounds.

The invention concerns combined pharmaceutical preparations which inhibit the abnormal growth of tumor cells. These combined preparations contain compounds as active substances which have glutaminase activity in combination with certain antineoplastic agents. In particular the invention concerns combined preparations of compounds having glutaminase activity and cytostatic compounds.

The generic term cancer embraces a large number of malignant diseases which are characterized by uncontrolled cell growth, lack of cell differentiation and penetration of neighbouring tissue and the formation of metastases. Almost any tissue can be the origin of such a malignant disease.

Current standard cancer therapies with antineoplastic agents involve considerable disadvantages and risks for the patients despite their advanced state of development. Due to their unspecific antiproliferative effect and the high doses, these antineoplastic agents do not only damage tumor cells but also healthy rapidly growing cells such as mucous membranes, haematopoietic cells (bone marrow) and hair follicles. Treatment with antineoplastic agents is usually associated with severe side-effects which impair the general well-being of patients (acute side-effects), it also results in irreversible damage to healthy tissue and increases the risk of secondary tumors. Furthermore, the tumors may become resistant to the agents which results in a loss of efficacy when they are used several times on a patient.

Several active substances are often combined and used simultaneously for treatment (polychemotherapy) in order to improve their effectiveness and reduce the development of resistance. However, despite this strategy the problems described above have not been satisfactorily solved. Thus new and gentler treatments are urgently needed to combat cancer for economic and medical reasons.

One possible approach for treating such malignant diseases is to reduce the glutamine concentration in the blood circulation. Glutamine is the most frequent amino acid in the blood circulation and plays a major role as a source of nitrogen and energy as well as a basic component for many endogenous syntheses. Tumor cells are especially dependent on glutamine from the blood circulation due to their strong growth.

In the 1980s numerous attempts were made to use glutamine-cleaving enzymes or reactive glutamine analogues for cancer treatment which remove the required glutamine from the tumors. Roberts et al. showed that Pseudomonas 7A glutaminase-asparaginase has an antineoplastic activity against a large number of leukaemia diseases in rodents, against ascites tumors and certain solid tumors (DE 41 40 003 A1 and WO 94/13817 A1). In addition it was determined in animal experiments with athymic mice that a combination of glutamine analogues (e.g. 6-diazo-5-oxo-L-norleucine (DON)) and glutaminase has a strong inhibitory action on human colon, breast and lung carcinomas (McGregor, W & Roberts, J (1989): Proc. Amer. Assoc. Cancer Res. 30, 578). In addition it was shown that treatment with glutaminase delayed the development of resistance to methotrexate (Roberts, J., Schmid, F. A. & Rosenfeld, H. J. (1979): Cancer Treat. Rep. 63: 1045-1054).

The initial promising animal experiments have, however, not yet led to marketable pharmaceutical preparations since all therapeutic approaches with glutaminase or glutamine analogues (e.g. DON, acivicin) had to be discontinued initially due to strong toxic side-effects (Medina M A (2001), Glutamine and Cancer, The Journal of Nutrition, vol. 131 (9): 2539S-42S). Despite the ideal principle of action of a glutamine depletion therapy, no therapy based on proteins having glutaminase activity has so far been successful.

Since, however, cancer diseases can to date only be inadequately treated it would be of major medical and economic importance to find a way to utilize the promising approach of a glutamine depletion therapy in the future.

The object of the present invention was therefore to increase the effect of antineoplastic agents and to provide preparations that can be used in concentrations which cause no or only slight toxicity and antibody formation.

It was surprisingly found that certain well-known antineoplastic agents in combination with compounds having glutaminase activity are suitable for achieving this object. The combinations act synergistically and are directly or indirectly toxic for dividing cells and can thus be used for an antineoplastic therapy. The components having glutaminase activity act as amplifiers which lower the required dose of antineoplastic agents and reduce the side effects as well as the late sequelae. Platinum complexes and in particular cis-platinum, oxaliplatinum, carboplatinum or derivatives thereof or anthracyclines and in particular doxorubicin or daunomycin or derivatives thereof are used as antineoplastic agents.

The invention concerns in particular combined preparations of compounds having glutaminase activity and cytostatic compounds. Cytostatic agents have already for a long time been a recognized and widely used treatment concept in antineoplastic therapy. They are used to destroy malignant cells having an uninhibited growth behaviour. Normal and healthy cells should be damaged as little as possible.

It was surprisingly found that compounds which have glutaminase activity have a synergistic effect in combination with antineoplastic agents. Thus in the case of cytostatic agents used according to the invention it was found that an amplification of the antiproliferative or antitumoral effect occurred.

In the sense of the present invention compounds which have glutaminase activity are understood as the proteins or enzymes: glutaminase, glutaminase-asparaginase, glutaminase analogues, derivatives and modifications thereof which either occur naturally or are produced synthetically and inhibit glutamine production. In a variant of the embodiment the compounds can be modified or provided with protective substances. Compounds modified with polyethylene glycol are preferably used. Glutaminase produced by genetic engineering or/and Pseudomonas glutaninase are particularly preferred. Glutaminases which are preferred according to the invention are described in WO 94/13817.

Antineoplastics are understood as substances which are suitable for and are used to damage or destroy microorganisms, parasites or tumor cells. These include in particular cytostatic agents or derivatives thereof from the following groups:
1. Alkylating and cross-linking compounds: They irreversibly damage DNA and include nitrogen mustard derivatives such as cyclophosphamide, ifosfamide, N-nitroso compounds such as carmustine, ethyleneimine, (aziridine) derivatives such as thiotepa, methane sulfonates such as busulfan, platinum complexes such as cis-platinum, oxaliplatinum or carboplatinum and in addition procarbazine, melphalan etc.
2. Antimetabolites: They displace natural metabolic building blocks; for example folic acid antagonists such as methotrexate, nucleoside analogues such as mercaptopurine, fluoruracil etc.
3. Mitosis inhibitors: They inhibit the assembly or degradation of the nuclear spindles, in particular vinca alkaloids (for example vincristine, vinblastine) and taxanes (for example paclitaxel).
4. Cytostatic antibiotics: Anthracyclines (e.g. daunorubicin, doxorubicin), bleomycin and mitomycin damage the cell by among others intercalation into DNA and inhibition of topoisomerases (e.g. etaposide) as well as actinomycins e.g. actinomycin D and mitoxantrone.
5. Hormones and hormone antagonists: They are used for tumors whose growth is hormone dependent; they include (anti)oestrogen (e.g. tamoxifen) and also aromatase inhibitors such as formestane, gestagens and antiandrogens such as flutamide.

Basically all these said compounds can be used together with a compound having glutaminase activity to produce combined preparations.

In addition to the combined preparations, the present invention concerns the use of antineoplastic agents together with compounds having glutaminase activity to treat cancer and other diseases that are associated with abnormal cell proliferation.

Such combinations of active substances are composed in particular of a glutaminase-asparaginase, preferably Pseudomonas 7A glutaminase-asparaginase and one or more antineoplastic agents from the above-mentioned groups.

Combinations of this invention are characterized by the fact that the antitumoral effect of conventional antineoplastic agents is significantly amplified by combining them with a compound having glutaminase activity and that the active protein ingredient can itself be used in concentrations that do not cause any toxic effects.

According to the invention the combined preparations can be used for cancer therapy. In particular it is possible to use subtherapeutic doses of a compound having glutaminase activity and antineoplastic agents which synergistically inhibit tumor cell growth. This means that the combination of active substances has a substantially higher antineoplastic activity than an active substance of this class of active substances would have alone.

Antineoplastic agents that are suitable for a combination include according to the invention platinum complexes and in particular cis-platinum, oxaliplatinum, carboplatinum or derivatives thereof, as well as anthracyclines e.g. actinomycin D, mitoxantrone and in particular the DNA intercalators doxorubicin and daunomycin (daunorubicin). Other antineoplastic agents that can be used for a combination include the DNA-alkylating agents clyclophosphamide, ifosfamide, melphalane, the antimetabolites methotrexate, 5-fluoruracil, the spindle or microtubuli toxins vincristine, vinblastine, paclitaxel; the topoisomerase inhibitor etoposide; the antibiotics actinomycin D, mitomycin, mitoxantrone, the hormones tamoxifen and flutamide.

The advantage of using a combined therapy with the aid of the pharmaceutical preparations of the present invention is the synergistic amplification of the antitumoral efficacy of the individual substances. This also allows a reduction of the doses and thus of the toxicity of the individual substances while at the same time retaining the antitumoral effectiveness when combining the individual substances. A combination treatment comprising the above-mentioned individual therapy principles also enables cytostatic resistances to be overcome in which case resistances to groups of substances as well as multiple resistances (pleiotropic cytostatic agent resistance) come into question.

Without wanting to be tied down to one theory, it is assumed that the synergistic effect observed according to the invention is based on the following mechanism of action. Alkylating cytostatic agents such as platinum preparations have an effect on cancer cells especially during cell division. Energy depletion due to glutamine removal by glutaminase as well as the absence of glutamine for DNA synthesis due to glutaminase results in a prolongation of the cell division time and thus to a prolongation of the phase in which the cancer cells are vulnerable to alkylating substances. It is well known (for example from tissue culture investigations) that cancer cells only start to grow in a medium above a certain concentration of glutamine. Furthermore, flow cytometry has shown that acute myeloid leukaemia responds particularly well to cytostatic treatment in the early hours of the morning. In addition the removal of glutamine in the cell as well as outside deprives the cancer cell of an important antioxidant which further contributes to its vulnerability to cytostatic agents.

When using the combined therapy it is possible to administer the active substances in a so-called fixed combination i.e. in a single pharmaceutical formulation which contains both active substances or to select a so-called free combination in which the active substances can be applied simultaneously or also successively in the form of separate pharmaceutical formulations.

If the active substances are solids, the active substances can be processed into solid pharmaceutical preparations by conventional processes in which for example both active substances are mixed together and pressed for example into tablets together with common carrier substances or auxiliary substances. It is, however, also possible to provide the active substances separately in a packaging unit that is ready for sale where the packaging unit contains the two active substances in separate pharmaceutical formulations.

If the active substances are provided in the form of injection solutions, the injection solutions can already contain the relevant combinations of active substances in a dissolved form that is ready to be injected. Basically it is, however, also possible to provide a parenteral formulation for each relevant active substance in a packaging unit such that the injection solutions can optionally be administered separately from one another. This form of application is the preferred method when the active substances are incompatible with one another.

In the case of a parenteral form of administration the active substances can also be present in bulk for example in a lyophilized form, optionally together with common pharmaceutical auxiliary substances and be reconstituted or solubilized by adding common pharmaceutical injection media.

The pharmaceutical preparations can be used in a liquid or solid form for enteral or parenteral administration. In this connection all conventional forms of administration come into consideration such as tablets, capsules, dragees, syrups, solutions and suspensions. Water is preferably used as an injection medium which contains the usual additives for injection solutions such as stabilizers, solubilizers and buffers. Such additives are for example tartrate and citrate buffer, ethanol, complexing agents such as ethylenediamine tetraacetic acid and non-toxic salts thereof as well as high-molecular polymers such as liquid polyethylene oxide to regulate the viscosity. Liquid vehicles for injection solutions must be sterile and are preferably filled into ampoules. Solid vehicles are for example starch, lactose, silicic acids, higher molecular fatty acids such as stearic acid, gelatin, agar-agar, calcium phosphate, magnesium stearate, animal and vegetable fats, solid high-molecular polymers such as polyethylene glycols; preparations suitable for oral administration can if desired contain flavouring agents or sweeteners.

The dosage depends on various factors such as mode of administration, species, age and individual condition. The doses that are to be administered daily are 0.005-100 mg/kg body weight per individual component.

In the combined preparations the ratio of the active substances can vary over a very wide range. Thus for example molar ratios between 1:10 to 1:1000 and 10:1 to 1000:1 are possible depending on the efficacy of the relevant active substances. In the case of a combination with cytostatic agents a ratio between 1:100 and 100:1 is preferred.

In a particularly preferred embodiment the present invention concerns a combined pharmaceutical preparation comprising at least one compound having glutaminase activity and at least one platinum complex and in particular cis-platinum. It was found that platinum complexes in a combination with glutaminase and in particular pseudomonas glutaminase exhibit synergistic effects on various tumor cell lines in tissue cultures of up to a factor of 120. The dose of cis-platinum and glutaminase can consequently each be considerably reduced compared to the doses required for single treatments. Thus the therapeutic dose for glutaminase in the combined preparations according to the invention is preferably 50-150 I.U./m² and in particular 100-130 I.U./m². The dose of platinum complex and in particular cis-platinum in the combined preparations according to the invention is preferably 1-20 mg/m² and in particular 2-15 mg/m² and even more preferably 5-10 mg/m². With such dosages solid tumors are already observed to respond after five days administration. The dose for a three week administration is preferably 10-100 mg/m² and in particular 20-50 mg/m².

In the case of combined preparations comprising a glutaminase and an anthracycline, in particular doxorubicin, the dose of glutaminase is again preferably 50-150 I.U./m² and in particular 100-130 I.U./m² body surface. The amount of doxorubicin is advantageously 1-20 mg/m² and in particular 2-15 mg/m² and even more preferably 5-10 mg/m² body surface for a once weekly administration. In the case of a three week administration the preferred dose is 5-60 mg/m² and in particular 10-50 mg/m² and even more preferably 15-30 mg/m² body weight.

The following examples document the synergistic effect of some representative combined preparations.

### EXAMPLE 1

In Vitro Tests for Investigating the Antitumoral Effect of Active Substances

The anti-tumoral effect of substances was examined in an in vitro cell culture test by means of the sulforhodamine method (Boyd M R, The NCI In Vitro Anticancer Drug Discovery Screen. In: Anticancer Drug Development Guide: Preclinical Screening, Clinical Trails and Approval (Teicher B. and Totowa N Publ.) 1985-1995; Skehan P et al., (1990), New Clorimetric Assay for Anticancer Drug Screening, J. Natl. Can. Instit. 82: 1107-1112). Cell lines from tumors of the breast (MCF7), lung (NCI-H460, A549), colon (SW-60, HT29) and CNS (SF-539) were used for the test. The tumor cells were cultured in RPMI 1640 medium containing 7.5% foetal calf serum at 37° C. and 5% CO₂. After the cells had grown for 24 hours, they were incubated with the substances to be tested for 48 hours. Mixtures without active substances served as controls and the blank value was determined before adding the active substances.

The antineoplastic agents used for the experiments were obtained from Sigma in cell culture quality.

A Pseudomonas 7A glutaminase-asparaginase modified with polyethylene glycol (DE 41 40 003 A1, WO 94/13817 A1 and WO 02/31498 A2) was used as the glutaminase.

### EXAMPLE 2

Determination of Synergistic Effects

After a 48 hour incubation period cell growth was determined by means of the absorption of the bound sulforhodamine dye at 575 nm. The percentage growth was calculated as follows:

$$PW = \frac{( {T}_{t} - {T}_{0} )}{( C - {T}_{0} )} \cdot 100$$

in which
PW denotes percentage growth,
C denotes untreated control cells,
T denotes the amount of treated cells
and the indices
0 and t denote the amount of cells at time 0 and after 48 hours.

### EXAMPLE 2a

Combination of Mitomycin and Glutaminase

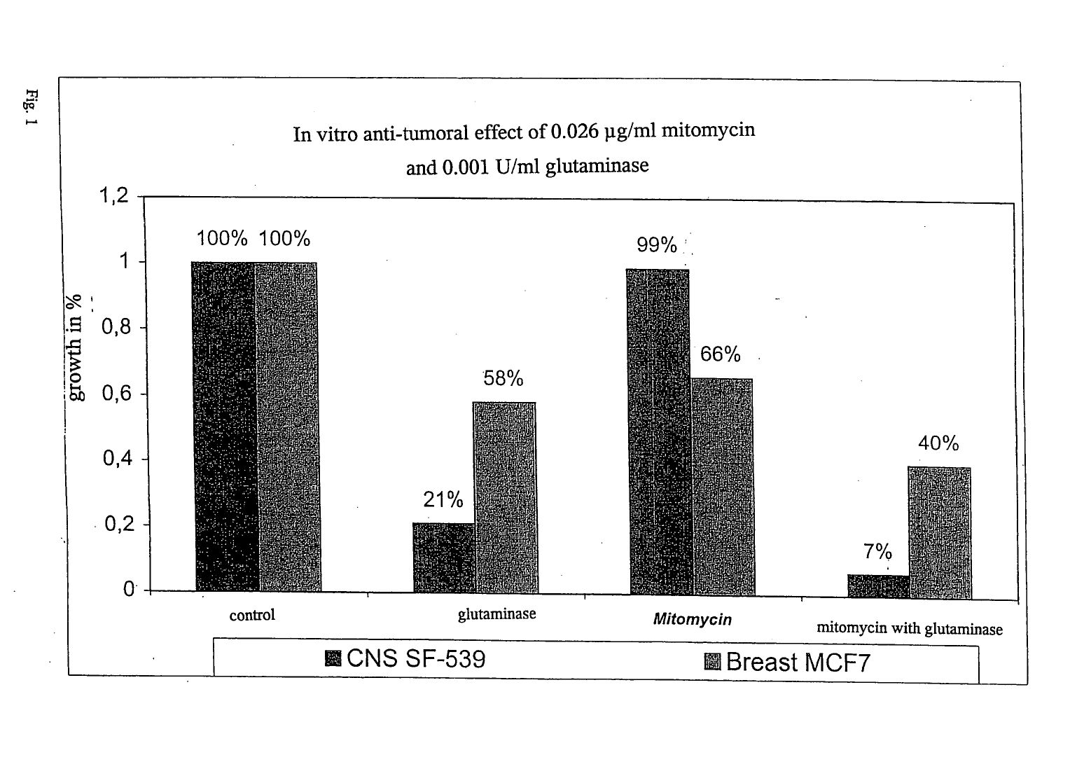

The anti-tumoral effect of 0.026 μg/ml mitomycin on cells of a CNS tumor (SF-539) and breast (MCF7) tumor alone and in combination with 0.001 U/ml glutaminase is shown in FIG. 1. Mitomycin alone has no effect on CNS cells; in combination with glutaminase the growth is reduced by 7% in comparison with the control. Mitomycin reduces the growth of cells of the breast tumor MCF7 to 66% compared to the control. The growth is reduced to 40% by combination with 0.001 U/ml glutaminase.

### EXAMPLE 2b

Combination of Mitoxantrone and Glutaminase

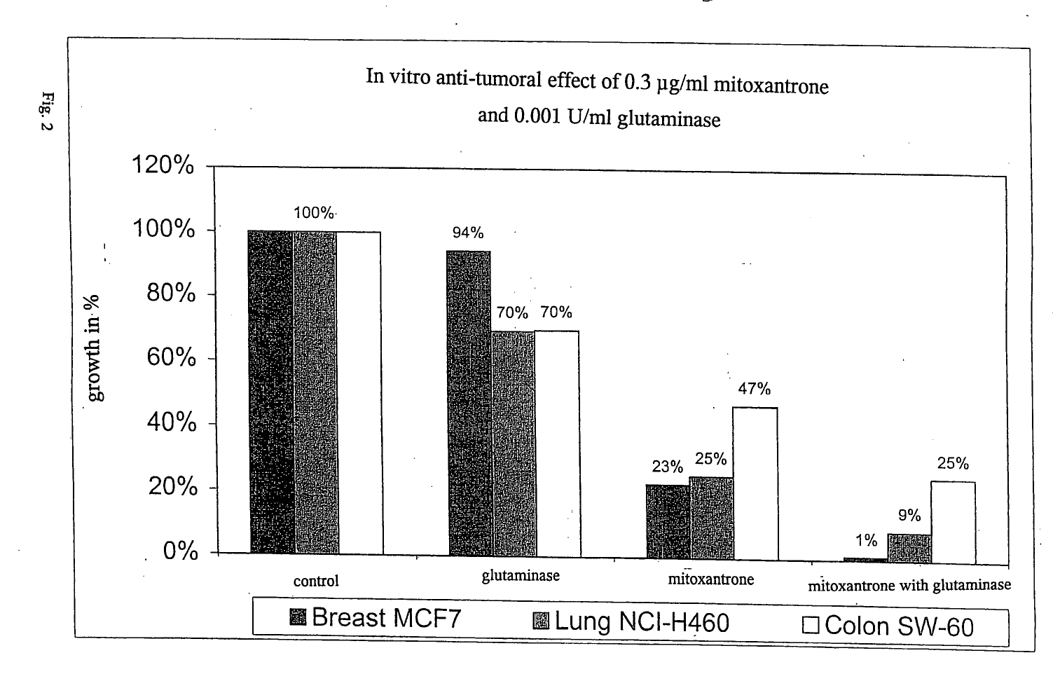

The anti-tumoral effect of 0.3 μg/ml mitoxantrone on cells of a breast (MCF7), lung (NCI-H460) and colon (SW-60) tumor alone and in combination with 0.001 U/ml glutaminase is shown in FIG. 2. Glutaminase alone only has a slight effect on the three tumors. Mitoxantrone alone reduces the tumor cell growth to 23% to 47%. In the combination growth of cells of the breast, lung and colon tumor is reduced to 1%, 9% and 25% respectively.

### EXAMPLE 2c

Combination of Cis-Platinum and Glutaminase

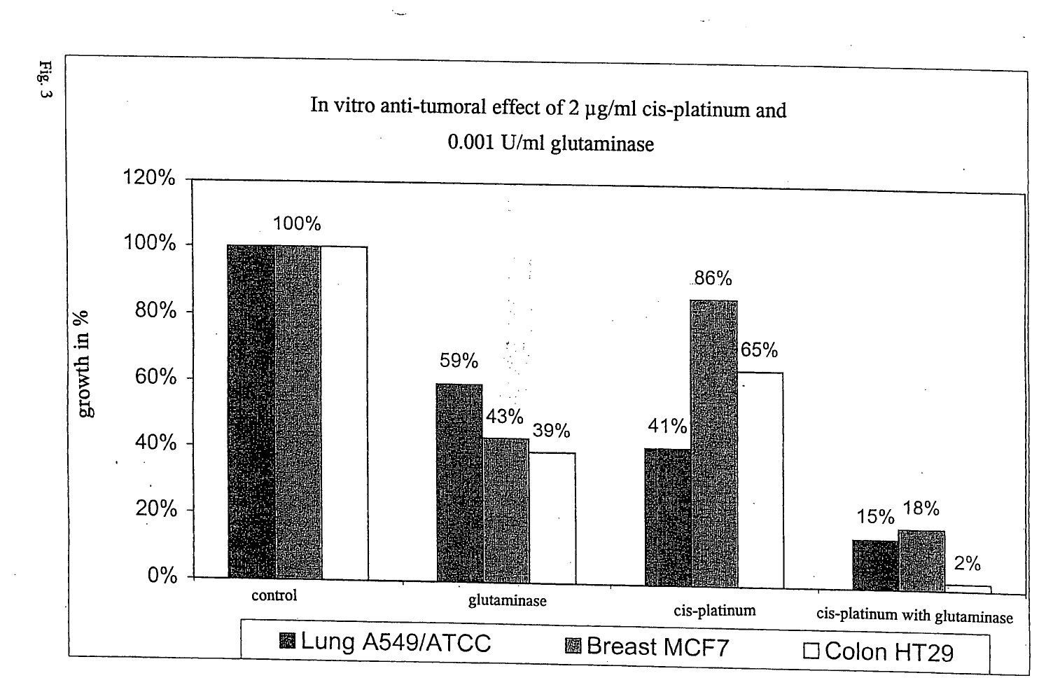

The anti-tumoral effect of 2 μg/ml cis-platinum on the cells of a lung (A549), breast (MCF7) and colon (HAT29) tumor alone and in combination with 0.001 U/ml glutaminase is shown in FIG. 3. Cis-platinum alone reduces the growth to 41%, 86% and 65% respectively. In combination with glutaminase the growth of the tumor cells is reduced to 15%, 18% and 2% respectively.

### EXAMPLE 2d

Combination of Etoposide and Glutaminase

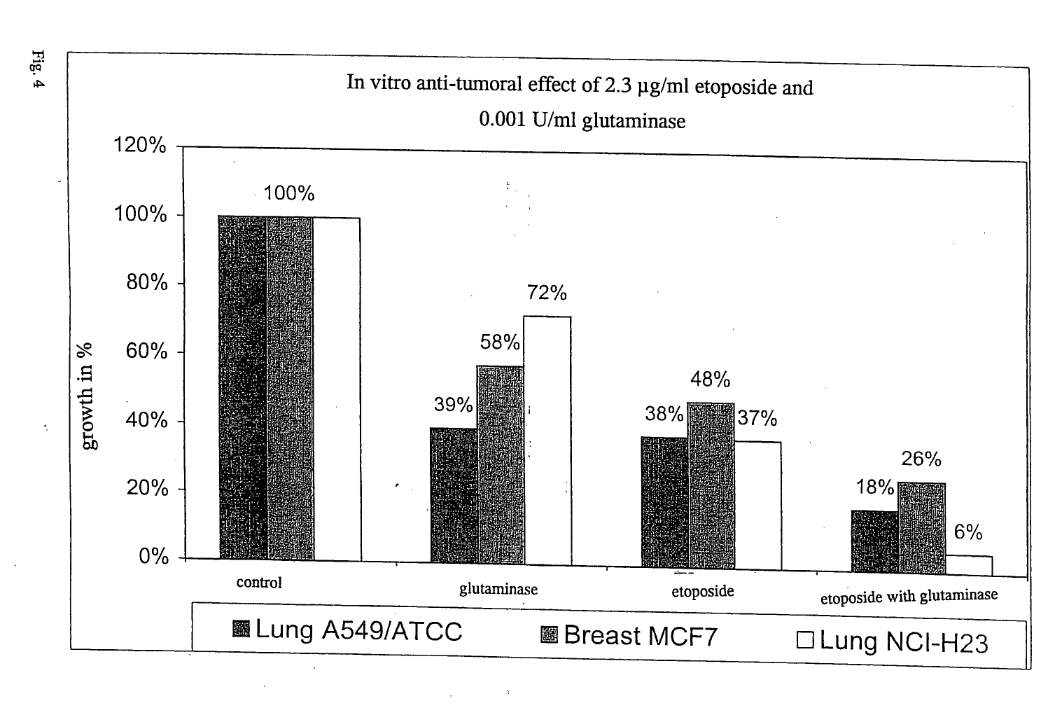

The anti-tumoral effect of 2.3 μg/ml etoposide on the cells of a lung (A549 and NCl-1-123) and breast (MCF7) tumor alone and in combination with 0.001 U/ml glutaminase is shown in FIG. 4. Etoposide alone reduces the growth of these cells to about 40% compared to the control. In combination with glutaminase the growth of the tumor cells is reduced to 18% and 6% and 26% respectively.

### EXAMPLE 2e

Combination of Melphalan and Glutaminase

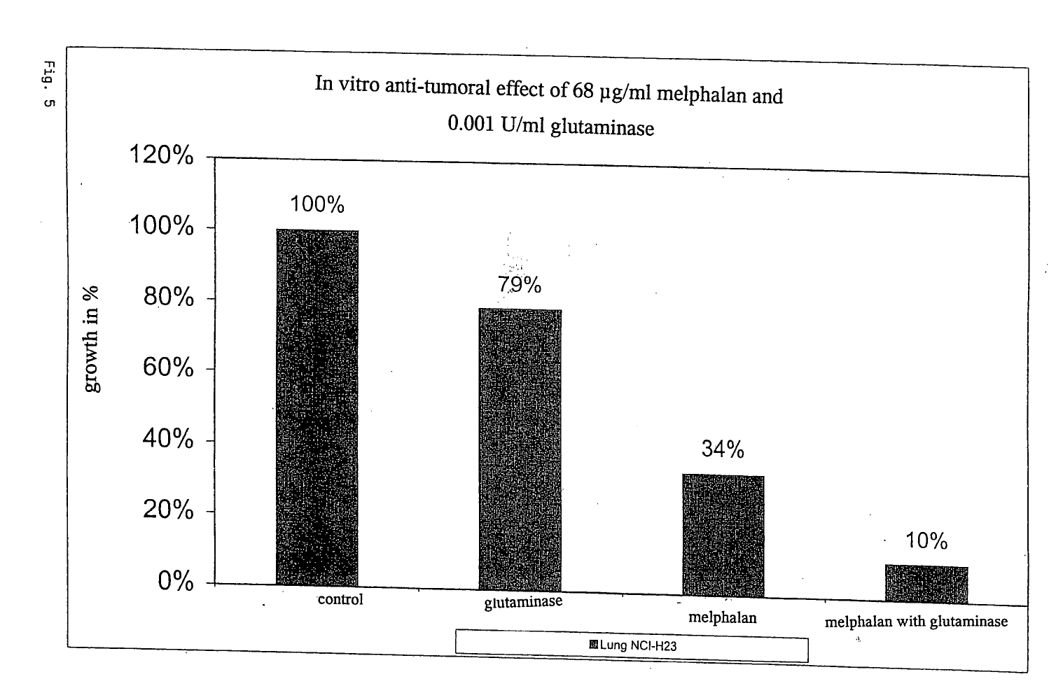

The anti-tumoral effect of 68 μg/ml melphalan on the cells of a lung (NCI-H23) tumor alone and in combination with 0.001 U/ml glutaminase is shown in FIG. 5. Melphalan reduces the tumor growth to 34%. In combination with glutaminase the growth is reduced to 10%.

1. Combined pharmaceutical preparation for cancer therapy comprising as active substances
a) at least one compound having glutaminase activity and
b) at least one antineoplastic agent selected from platinum complexes and anthracyclines.

2. Preparation as claimed in claim 1,
characterized in that
the compound having glutaminase activity is a glutaminase, glutaminase-asparaginase, glutaminase analogue, derivative or modification of the same and is either of natural origin or is produced synthetically.

3. Preparation as claimed in claim 2,
characterized in that
the compound having glutaminase activity is from Pseudomonas and is preferably Pseudomonas 7A glutaminase-asparaginase.

4. Preparation as claimed in one of the claims 1 to 3,
characterized in that
the compound having glutaminase activity is modified preferably with polyethylene glycol.

5. Preparation as claimed in one of the claims 1 to 4,
characterized in that
it comprises doxorubicin, daunomycin, actinomycin D or/and mitoxantrone.

6. Preparation as claimed in one of the claims 1 to 5,
characterized in that
it comprises cis-platinum, oxaliplatinum or/and carboplatinum.

7. Process for producing pharmaceutical preparations as claimed in one of the claims 1 to 6,
characterized in that
the active substances optionally together with common pharmaceutical carrier substances or auxiliary substances are mixed and processed into oral or parenteral forms of administration.

8. Use of in particular a compound having glutaminase activity and at least one antineoplastic agent selected from platinum complexes and anthracyclines to produce an agent for an antineoplastic therapy.

9. Method for treating cancer and other diseases which are associated with abnormal cell proliferation,
characterized in that
at least one compound having glutaminase activity and at least one antineoplastic agent selected from platinum complexes or anthracyclines are administered in a molar ratio between 1:10 to 1:1000 and 10:1 to 1000:1, where the doses to be administered daily are 0.005-100 mg/kg body weight per individual component.
